(19) United States
(12) Patent Application Publication
(10) Pub. No.: US 2006/0178312 A1
(43) Pub. Date: Aug. 10, 2006

(54) Process for the production of melagatran

(75) Inventors: Marcus Grehn, Sodertalje (SE); Tibor Musil, Sodertalje (SE); Magnus Sjogren, Sodertalje (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/561,944

(22) PCT Filed: Jun. 23, 2004
(86) PCT No.: PCT/SE04/01016

§ 371 Date: Dec. 21, 2005

(30) Foreign Application Priority Data
Jun. 25, 2003 (SE) 0301879-3

(51) Int. Cl. A61K 38/05; C07K 5/06; C07D 205/02

(52) U.S. Cl. 514/18; 530/331; 548/953

(57) ABSTRACT

There is provided a process for the production of melagatran, Formula (I) ex vivo, which process comprises the hydrolysis of a compound of Formula II, wherein R is C₁₋₆ alkyl or a benzylic group, to form, in substantially salt-free form, an intermediate compound of formula II followed by reduction of that intermediate.

### FIELD OF THE INVENTION

This invention relates to a novel process for the production of the thrombin-inhibiting compound, melagatran.

### PRIOR ART

International patent application WO 94/29336 discloses a group of compounds that are useful as inhibitors of serine proteases, such as thrombin and/or kininogenases. The thrombin-inhibiting compounds are thus indicated as anticoagulants, and the kininogenase-inhibiting compounds as anti-inflammatory agents.

One of the thrombin-inhibiting compounds that is specifically disclosed in WO 94/29336 is HO₂C—CH₂—(R)Cgl-(S)Aze-Pab-H (wherein Cgl represents cyclohexylglycinyl, Aze represents azetidine-2-carboxyl, and Pab represents para-amidinobenzylamino), which is also known as melagatran (see Example 1 of WO 94/29336).

International patent application WO 97/23499 discloses prodrugs of inter alia melagatran. Prodrugs described in WO 97/23499 that may be mentioned include those of the formula RO₂C—CH₂—(R)Cgl-(S)Aze-Pab-OH, wherein R represents linear or branched C₁₋₆ alkyl (e.g. C₁₋₄ alkyl, especially methyl, n-propyl, i-propyl, t-butyl and, particularly, ethyl) or benzyl, the OH group replaces one of the amidino hydrogens in Pab, and Cgl, Aze and Pab are as defined above. The prodrug of the formula EtO₂C—CH₂—(R)Cgl-(S)Aze-Pab-OH (see Example 17 of WO 97/23499), which is also known as ximelagatran, is now in full clinical development for use in oral delivery to patients.

The synthetic routes that are described for melagatran and ximelagatran in the respective above-mentioned patent applications are quite different.

We have now found that melagatran may be prepared directly in a cost-effective and convenient manner from certain alkyl ester derivatives that are disclosed in WO 97/23499, including ximelagatran itself.

### DESCRIPTION OF THE INVENTION

According to a first aspect of the invention there is provided a process for the production of melagatran, 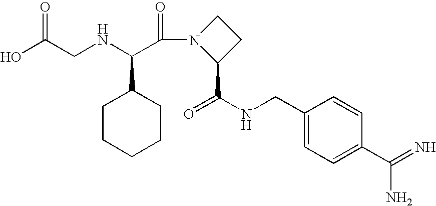
ex vivo, which process comprises the hydrolysis of a compound of formula I, 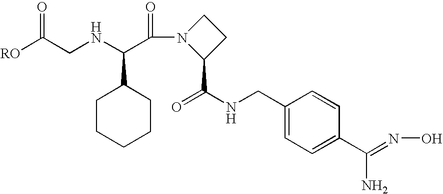
wherein R represents linear or branched C₁₋₆ alkyl or a benzylic group, to form, in substantially salt-free form, an intermediate compound of formula II, 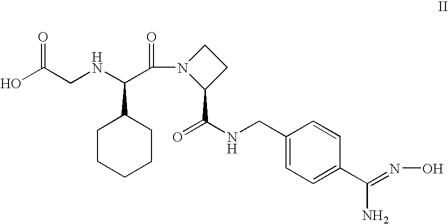
followed by reduction of that intermediate compound, which process is referred to hereinafter as “the process of the invention”.

Preferred values of the substituent R include C₁₋₄ alkyl, such as C₁₋₃ alkyl, particularly methyl, n-propyl, i-propyl and especially ethyl groups, or benzylic groups such as optionally substituted benzyl. Suitable optional substituents on benzyl groups include halo (e.g. chloro and bromo), C₁₋₆ (e.g. C₁₋₄) alkyl (such as methyl), and C₁₋₆ (e.g. C₁₋₄) alkoxy (such as methoxy).

By “substantially salt-free”, we mean that the intermediate compound of formula II is formed, and thus may be isolated (for example by precipitation), following the hydrolysis step and prior to the reduction step of the process of the invention, in >95%, such as >98%, preferably >99%, and particularly >99.9%, free acid (and/or any Zwitterionic) form (i.e. no more than 5%, such as 2%, preferably 1% and particularly 0.1% w/w, respectively, of compound of formula II is in the form of a salt (with either an inorganic or an organic counter-ion)).

Compounds of formula I may be prepared by way of known techniques, for example as described in international patent application WO 97/23499.

The hydrolysis step may or may not be carried out under basic conditions (for example, hydrolysis may also be carried out under acid conditions). Base hydrolysis may be conducted in the presence of an alkali metal carbonate, such as potassium carbonate or sodium carbonate or, preferably, an alkali metal hydroxide, such as lithium hydroxide, potassium hydroxide or, preferably, sodium hydroxide.

Base may be added in solid form, but is preferably added in the form of an aqueous solution (such as a 1M to 3M (e.g. 2M) aqueous solution) to a solution of a compound of formula I in an appropriate solvent, for example a water-miscible solvent, such as a lower alkyl alcohol (e.g. a C₁₋₆ alkyl alcohol, such as i-propanol, methanol or, particularly, ethanol), a diol (such as ethylene glycol), or an ether (such as tetrahydrofuran, dioxane and/or a dimethylglycolate), and/or water. Mixtures of these solvents may also be employed. Alternatively, the hydrolysis step may be performed in a two-phase system comprising an organic solvent that is inert to hydrolysis, such as toluene, and an aqueous solution of one or more of the bases described hereinbefore.

The hydrolysis may be carried out at between 0° C. and 100° C. depending upon the boiling point of the solvent that is employed. The reaction is, however, preferably carried out at around room temperature or above (e.g. between about 15° C. and 50° C. or thereabouts). Reaction times are in the range of about 15 minutes to about 6 hours, such as about 30 minutes to about 4 hours. The skilled person will appreciate that the reaction time will depend upon inter alia the temperature of the reaction mixture as well as the solvent that is employed.

Compounds of formula II may be formed (and thus isolated) in substantially salt-free form by way of preparative work-up which involves acidification of the reaction mixture when the hydrolysis step is carried out under basic conditions. Acidification may be conducted by addition of an inorganic acid, such as sulphuric acid, phosphoric acid, hydrobromic acid or, preferably, hydrochloric acid. The acid may be added as such, but is preferably provided in the form of an aqueous solution. The pH value of the resultant mixture should preferably be adjusted to a weakly acidic pH, such as pH 4 to 6, preferably pH 4.5 to 5.5, and especially pH 5 or thereabouts.

We have found that, by performing preparative work up in the above-mentioned manner, salts, such as inorganic salts, that are formed conveniently dissolve in an aqueous phase prior to separation from the intermediate. However, the hydrolysis step may also be performed in the presence of a water-free base, and work up subsequently performed in the presence of a water-free acid, with a view to providing the intermediate in a form in which it is dissolved in a suitable solvent, and wherein any inorganic salts that are formed precipitate and are removed by filtration.

Irrespective of the technique employed for forming the intermediate compound of formula II in substantially salt-free form, it may be isolated if desired via an appropriate technique, for example by solvent evaporation (in the case where the intermediate is formed in a form in which it is dissolved in a suitable solvent), or preferably by precipitation and filtration (in the case where unwanted salts are dissolved in an aqueous phase prior to separation).

The reduction step of the process of the invention is preferably carried out by way of hydrogenation in the presence of a suitable catalyst system (i.e. a hydrogenolysis reaction). The catalyst is preferably a precious transition metal, for example platinum, ruthenium or, especially, palladium. The metal can be used as such in powder form, as its oxide or hydroxide or, preferably, on a suitable support, such as powdered charcoal. Typically, palladium on charcoal is used (e.g. 5% Pd/C).

Hydrogenation may be carried out in the presence of an appropriate solvent system. The solvent system that is employed is done so with a view to enhancing the solubility therein of the intermediate compound formed following the previous step. In this respect, the amount of water in any alcohol:water mixture is preferably in the range 20% (e.g. 25%) to 45% v/v and more preferably in the range 30% to 40% v/v. Appropriate solvent systems include lower alkyl alcohols (e.g. C₁₋₆ alkyl alcohols, such as i-propanol, methanol or, particularly, ethanol) and/or water. Preferred solvent systems include mixtures of the above-mentioned lower alkyl alcohols (particularly methanol and, more particularly, ethanol) and water in appropriate proportions. For example, when the solvent system is a mixture of methanol and water, appropriate mixtures are in the range 75:25 to 65:35, more preferably 72:28 to 67:33, such as 70:30 (methanol:water; v/v) or thereabouts; and when the solvent system is a mixture of ethanol and water, appropriate mixtures are in the range 70:30 to 60:40, more preferably 65:35 to 61:39, such as 62.5:37.5 (ethanol:water; v/v) or thereabouts.

Hydrogenation may be carried out under a positive pressure of hydrogen (e.g. at least 2 bar, such as at least 3 bar and, preferably at least 4 bar of hydrogen pressure). Reactions may be carried out at an appropriate reaction temperature, such as at elevated temperature, depending upon the solvent system that is employed. When the solvent system is a methanol:water mixture (for example in proportions in the region 70:30 v/v), typical reaction temperatures are in the range 55° C. to 65° C. When the solvent system is an ethanol:water mixture (for example in proportions in the region 60:40 to 65:35 (e.g. 62.5:37.5 v/v)), typical reaction temperatures are in the range 65° C. and 75° C., such as between 65° C. and 72° C., for example 68° C. to 70° C. or thereabouts. Typical reaction times are in the region of between 12 and 48 hours, such as 18 to 36 hours e.g. between 20 and 30 (such as 24) hours or thereabouts. The skilled person will also appreciate that the nature and the amount of catalyst will have an effect on the rate of reaction and therefore reaction times.

Preparative work-up following reduction may be carried out using known techniques, for example by cooling to ambient temperature, filtration and evaporation of solvents, for example as described hereinafter.

It is a further preferred embodiment of the invention that the hydrogenation is carried out in the absence of inorganic acids and/or additional carboxylic acids. We have found, surprisingly, that, when the reduction step of the process of the invention is carried out in the absence of such acids, the reduction proceeds efficiently and results in melagatran in a form in which it does not necessarily need to be purified in order to remove salts formed during the reaction or during preparative work up. By “absence of inorganic acid/additional carboxylic acid”, we mean that the reaction mixture comprises less than 3%, such as less than 2%, preferably less than 1%, more preferably less than 0.5%, and especially less than 0.1% (w/w), of such acids (notwithstanding the essential presence of the reactant compound of formula II) originating from a separate and/or independent (i.e. exogenous) source.

Melagatran may thereafter be isolated and, if desired, purified by way of known techniques, such as by way of recrystallisation from an appropriate solvent system (e.g. as described in international patent application WO 01/02426), followed by decanting, filtering and/or centrifuging. Crystallisation can be effected with or without seeding.

Melagatran formed by way of the process of the invention may be utilised in the treatment and/or prophylaxis of conditions in which inhibition of thrombin is desired or required, including those conditions described in inter alia international patent applications WO 94/29336 and WO 97/23499.

The process of the invention has the advantage that melagatran may be prepared in higher yields, more quickly, more efficiently, in a higher purity, more conveniently, and/or at a lower cost, than when prepared by way of techniques described in the prior art for the total synthesis of melagatran.

The invention is illustrated, but in no way limited, by the following example.

### EXAMPLE 1

Synthesis of Melagatran Monohydrate

(a) HO₂C—CH₂—(R)Cgl-(S)Aze-Pab-OH

Ximelagatran (see Example 17 of WO 97/23499; 10 g; 21.11 mmol; 1 eq.) was dissolved in ethanol (100 mL) and 2M of NaOH solution (12.7 mL; 25.34 mmol; 1.2 eq.) was added. The mixture was stirred for four hours at 20-25° C. When the reaction was complete, the reaction mixture was acidified (to pH 5) with 2M HCl solution (12.7 mL; 25.34 mmol; 1.2 eq.), after which an additional 40 mL of water was added. The precipitated white solid was collected by filtration. After drying, the sub-title compound was obtained as an off-white solid. The yield was approximately 90% (w/w).

(b) Melagatran Monohydrate

HO₂C—CH₂—(R)Cgl-(S)Aze-Pab-OH (5 g; 11.22 mmol; see step (a) above) was mixed with 50 mL of ethanol:water (62:38) and palladium on carbon (5% Pd/C, 0.75 g; moist 50% w/w water). The resultant slurry was then hydrogenated (4 bar pressure of H₂ at 68° C.) under vigorous stirring for 24 hours. After cooling to room temperature, activated carbon (0.5 g) was added under an inert atmosphere and the mixture was stirred for 30 minutes. The catalyst and carbon were filtered off and the filtrate was evaporated to dryness. The title compound was obtained as a white solid (approximately 4.9 g).

Crude melagatran monohydrate may be recrystallized as described in international patent application WO 01/02426.

1. A process for the production of melagatran, 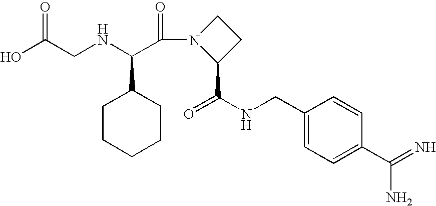
ex vivo, which process comprises the hydrolysis of a compound of formula I, 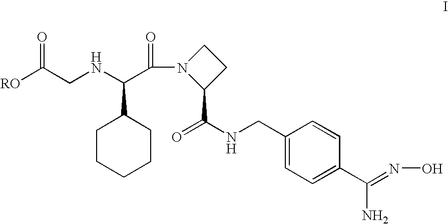
wherein R represents a linear or branched C₁₋₆ alkyl or a benzylic group, to form, in substantially salt-free form, an intermediate compound of formula II, 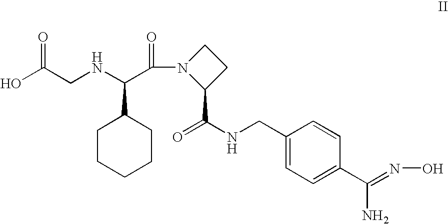
followed by reduction of the intermediate compound.

2. A process as claimed in claim 1, wherein R represents a linear or branched C₁₋₄ alkyl or a benzylic group.

3. A process as claimed in claim 2, wherein R represents methyl, ethyl, n-propyl, i-propyl or benzyl.

4. A process as claimed in claim 3 wherein R represents ethyl.

5. A process as claimed in claim 1 wherein the hydrolysis step is carried out in the presence of a base.

6. A process as claimed in claim 5 wherein the base is an alkali metal carbonate or an alkali metal hydroxide.

7. A process as claimed in claim 6 wherein the base is potassium carbonate, sodium carbonate, lithium hydroxide, potassium hydroxide or sodium hydroxide.

8. A process as claimed in claim 7 wherein the base is sodium hydroxide.

9. A process as claimed in claim 1 or claim 5, wherein the hydrolysis step is carried out in the presence of a lower alkyl alcohol, a diol, an ether, water or a combination thereof as solvent.

10. A process as claimed in claim 9, wherein the solvent is a mixture of a C₁₋₆ alkyl alcohol and water.

11. A process as claimed in claim 10, wherein the alcohol is ethanol.

12. A process as claimed in claim 1, wherein the hydrolysis step is carried out at between about 15° C. and about 50° C.

13. A process as claimed in claim 1, wherein a preparative work up follows the hydrolysis step and involves acidification of the reaction mixture.

14. A process as claimed in claim 13 wherein the acidification uses sulphuric acid, phosphoric acid, hydrobromic acid or hydrochloric acid.

15. A process as claimed in claim 13 wherein the pH is adjusted to a weakly acidic value.

16. A process as claimed in claim 15, wherein the pH value is about pH 5.

17. A process as claimed in claim 1, wherein the reduction step is carried out by hydrogenation in the presence of a suitable catalyst system.

18. A process as claimed in claim 17, wherein the catalyst is a precious metal.

19. A process as claimed in claim 18, wherein the metal is platinum, ruthenium or palladium.

20. A process as claimed in claim 19, wherein the metal is palladium.

21. A process as claimed in claim 18 wherein the metal is provided on a support.

22. A process as claimed in claim 21 wherein the support is powdered charcoal.

23. A process as claimed in claim 17, wherein the hydrogenation is carried out in the presence of a solvent system comprising a lower alkyl alcohol, water or a mixture thereof.

24. A process as claimed in claim 23 wherein the alcohol is a C₁₋₆ alkyl alcohol.

25. A process as claimed in claim 24, wherein the alcohol is i-propanol, methanol or ethanol.

26. A process as claimed in claim 25 wherein the solvent system is a mixture of methanol and water or ethanol and water.

27. A process as claimed in claim 26 wherein the solvent system is a mixture of methanol and water in proportions of about 70:30 (v/v), or a mixture of ethanol and water in proportions of about 62.5:37.5 (v/v).

28. A process as claimed in claim 17 wherein the hydrogenation is carried out at elevated temperature.

29. A process as claimed in claim 28, wherein the hydrogenation is carried out under a positive pressure of hydrogen.

30. A process as claimed in claim 29, wherein the hydrogenation is carried out under at least 4 bar of hydrogen pressure.

31. A process as claimed in claim 17 wherein the hydrogenation is carried out in the absence of an inorganic acid, or an additional carboxylic acid, as part of the reaction mixture.
